(19) United States
(12) Patent Application Publication
(10) Pub. No.: US 2018/0361152 A1
(43) Pub. Date: Dec. 20, 2018

(54) Multi-source stimulation

(71) Applicant: NewSouth Innovations Pty Limited, Sydney (AU)

(72) Inventors: Paul Brendon Matteucci, Cougee (AU); Gregg Jorgen Suaning, Lisarow (AU)

(21) Appl. No.: 15/998,793

(22) Filed: Aug. 16, 2018

Related U.S. Application Data
(62) Division of application No. 14/831,812, filed on Aug. 20, 2015, now abandoned.
Application No. 14/831,812 is a continuation-in-part of application No. 14/342,365, filed on Jul. 23, 2014, now Pat. No. 10,086,200.
(60) Provisional application No. 61/540,088, filed on Sep. 28, 2011.

(30) Foreign Application Priority Data
Aug. 31, 2011 (AU) 2011903509
Oct. 29, 2014 (AU) 2014904338

(51) Int. Cl. A61N 1/36; A61N 1/05

(52) U.S. Cl. CPC A61N 1/36046; A61N 1/36164; A61N 1/0543

(57) ABSTRACT

A system and method are described for stimulating excitable tissue. The system includes a monopolar stimulation source that generates a sub-threshold field in the vicinity of the excitable tissue, the sub-threshold field being below a threshold at which activation of the excitable tissue occurs. One or more local stimulation sources generate a local field, which in combination with the sub-threshold field exceeds the threshold of the excitable tissue.

### FIELD OF THE INVENTION

The present invention relates to systems and methods for electronic stimulation of tissue. In one form the invention relates to neural stimulation electrodes for retinal prostheses.

### BACKGROUND OF THE INVENTION

Retinal prosthetic devices may use electrode arrays to deliver electrical pulses to the retina in order to evoke patterned light perception. The electrodes evoke perception of phosphenes via remaining intact retinal neurons of vision-impaired users. One problem with implementing these electrode arrays is the trade-off between high density of electrodes providing better visual acuity in the implant recipient and the interference between adjacent stimulating electrodes. Consequently improved methods of implementing electrode arrays are desirable in order to effect neural stimulation through the elicitation of substantially discrete phosphenes.

Another trade-off involves the distance between the stimulating electrodes and the neurons targeted for activation. The amount of electric charge that is required from a given stimulation strategy in order to elicit a response from the neurons increases with distance and may eventually require more electric charge than may be safely, effectively or otherwise practically be delivered. Consequently improved methods of reducing the amount of electric charge delivered from each electrode are desirable in order to maintain the safe and efficacious operation of the neural stimulation.

The inventor has previously described systems and methods for implementing electrode arrays in the PCT application PCT/AU2012/001027 “Neural Stimulation Electrodes”, published as WO 2013/029111, the contents of which are hereby incorporated by reference.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

According to one aspect of the invention there is provided a system for stimulating excitable tissue, comprising:

a monopolar stimulation source that generates a first field in the vicinity of the excitable tissue; and

a local stimulation source that generates a local field, which in combination with the first field exceeds a threshold at which of the excitable tissue occurs.

According to a further aspect of the invention there is provided a neural prosthesis comprising:

an electrode array comprising a plurality of stimulating electrodes each having at least one associated bipolar return electrode; and

a monopolar return electrode;

a plurality of bipolar electrical return paths associated with the respective bipolar return electrodes; and

a monopolar electrical return path associated with the monopolar return electrode;

wherein, in use, the plurality of stimulating electrodes provide stimulating currents to the tissue of a recipient; and for at least one stimulating electrode a total return current is divided between a first current in the associated bipolar electrical return path and a monopolar current in the monopolar electrical return path.

According to a further aspect of the invention there is provided a method for stimulating excitable tissue, comprising:

generating, with a monopolar stimulation source, a sub-threshold field in the vicinity of the excitable tissue, the sub-threshold field being below a threshold at which activation of the excitable tissue occurs; and

generating a local field with a local stimulation source, wherein the local field in combination with the sub-threshold field exceeds the threshold of the excitable tissue.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a schematic cross-sectional view of an eye with an example of an implanted neural prosthesis.

FIG. 2 is a plan view of an example of an electrode array.

FIG. 3 is a schematic representation of the electrode array of FIG. 2 with a superimposed hexagonal logical array used for addressing.

FIG. 4 is the electrode array of FIG. 2 with the superimposed hexagonal array of FIG. 3 shifted by one position.

FIG. 5 shows a measured voltage topography resulting from the use of four stimulating electrodes with guard rings.

FIG. 6 shows a measured voltage topography resulting from the use of the same four stimulating electrodes as in FIG. 5 with single return paths through one each of the six electrodes surrounding each stimulating electrode.

FIG. 7A is a schematic representation of the electrical field resulting from the use of a guard ring configuration.

FIG. 7B is a schematic representation of the reshaped electrical field resulting from the use of a hybrid configuration.

FIG. 7C is a schematic representation of the electrical field resulting from the use of a single monopolar return path.

FIG. 8 shows a schematic diagram of an arrangement using a hybrid return path.

FIG. 9 shows a schematic diagram of the circuitry used to implement the hybrid return path of FIG. 8.

FIG. 10 illustrates experimental results showing the effects on stimulation threshold of different ratios of monopolar and hexapolar stimulation with the bars indicating the standard error.

FIG. 11 illustrates experimental results showing the effects on charge containment of different ratios of monopolar and hexapolar stimulation with the bars indicating the standard error.

FIG. 12A shows a schematic diagram of an arrangement in which hexapolar and monopolar contributions are generated from different sources.

FIG. 12B is a schematic illustration of the monopolar and hexapolar fields generated in the arrangement of FIG. 12A.

FIG. 13A shows a schematic diagram of a further arrangement in which hexapolar and monopolar contributions are generated from different sources.

FIG. 13B is a schematic illustration of the monopolar and hexapolar fields generated in the arrangement of FIG. 13A.

FIG. 14A is a schematic illustration of a longitudinal array of electrodes with tri-polar stimulation of target tissue.

FIG. 14B is a schematic illustration of the longitudinal array of FIG. 14A with a monopolar field

FIG. 14C illustrates the longitudinal electrode array of FIG. 14A with the tri-polar stimulation used in conjunction with a monopolar field.

### DETAILED DESCRIPTION OF THE EMBODIMENTS

In one application the present invention is applied to a retinal neuroprosthesis. In other applications described below the invention is applied in deep brain stimulation of the sub-thalamic nucleus or stimulation of the auditory system via the cochlea.

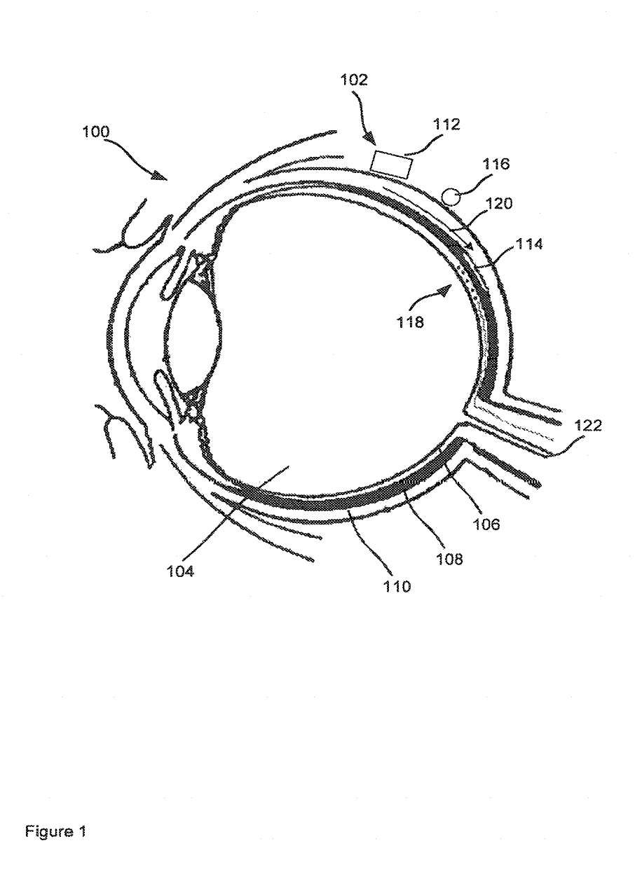

FIG. 1 shows a cross section of an eye 100 with the implanted portion of a retinal prosthesis 102. The eye 100 includes three layers bounding the vitreous humour 104: the neural retina 106, choroid 108 and sclera 110.

The prosthesis 102 includes at least one electronics capsule 112, an electrode array 114 and at least one monopolar return electrode 116. When implanting these components of the prosthesis the electrode array 114 is inserted into the eye to be near to the neurons 118 that lie in the neural retina 106 and that need to be stimulated. However, the choroid 108 is the vascular layer of the eye so that incisions may result in unwanted bleeding. Therefore, one method of inserting the electrode array 114 without penetrating the choroid 108 is to make an incision through the sclera 110, for example proximate the electronics capsule 112, and to slide the array along the interface between the sclera 110 and the choroid 108, for example in the direction of arrow 120 until the electrode array is in the desired location, adjacent the necessary neurons 118 but on the opposite side of the choroid 108. In this configuration stimulating pulses from the electrode array 114 may stimulate the neurons 118 from across the choroid. Thus, there is a physical distance between the electrode array 114 and the neurons 118. The electronics capsule 112 may be remote from the site of stimulation and connected to the electrodes by way of a multi-conductor lead wire, with one conductor per electrode. The configuration of FIG. 1 is merely an illustrative example of positioning within the recipient's orbit.

When signals are transmitted to the eye for neural stimulation, electrical impulses or stimuli are presented to the eye by injecting electrical current from the electrode array 114 into the tissue, and the current is returned to the implant circuitry via one or more of the electrodes in the array 114, and/or the monopolar return electrode 116. In this way the neurons 118 are stimulated so that they contribute to the perception of phosphenes. Information within the neurons 118 passes to the user's brain via the optic nerve 122.

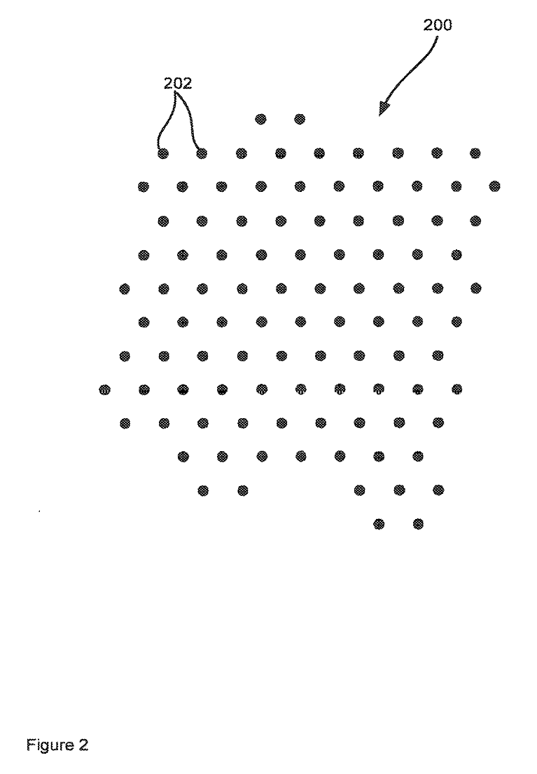

A high density of electrodes may provide a high density of phosphenes thereby allowing better visual acuity in the implant recipient. However, if any two regions of activation are too close, injected charge may interfere. Arranging individual electrodes 202 in a staggered geometric array 200 as shown in FIG. 2 allows for high density of phosphenes. When providing stimuli, the electrodes need to be addressed in some way to be able to provide the required stimulus.

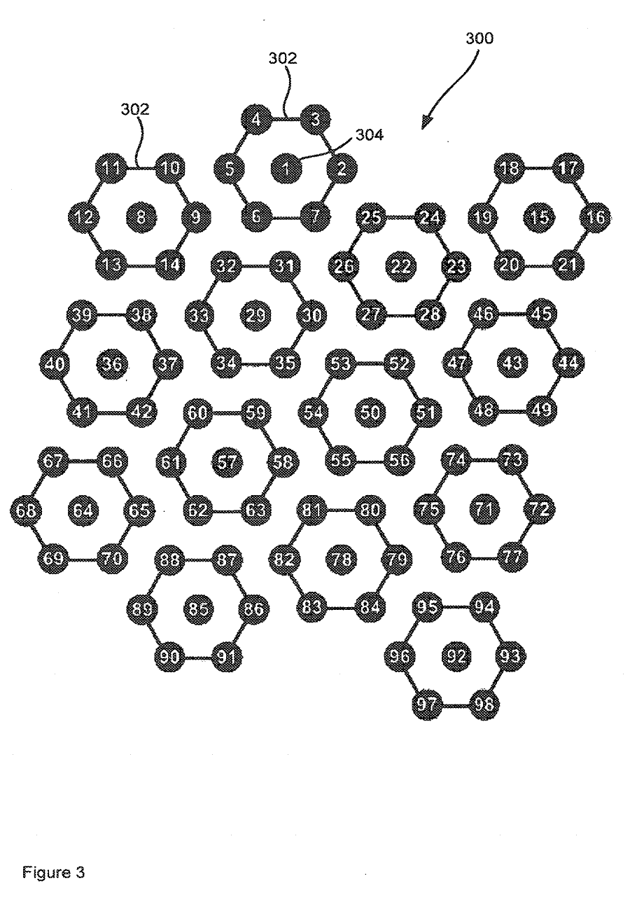

One method of addressing the electrodes, as described in US patent application number US2009/0287275, the contents of which are incorporated herein by reference, comprises using a superimposed logical array 300 as shown in FIG. 3. This scheme has the advantage of enabling individual electrodes to be addressed in parallel to facilitate parallel stimulation. Repeating regular patterns, here hexagonal shapes 302, are overlaid on the physical electrode array 200. Each of the hexagons 302 contains seven electrodes 202. A numbering scheme, for example that shown in FIG. 3, is used to specify the centre of each hexagon so that the centre of each hexagon is separated from the centres of the adjacent hexagons throughout the array. In the addressing scheme, a single stimulation identifier is used to specify the stimulating electrodes within a plurality of the hexagons. This provides an efficient system for addressing the electrode array.

The centre of each hexagon 302, for example electrode 304, serves as the stimulating electrode, and is associated with a power source that may be located in the electronics capsule 112. One, two or all of the immediately adjacent electrodes (the electrodes at the corners of the hexagons 302) and/or a distant monopolar return path electrode 116 serve as the electrical return path for the current stimulus. During the first phase of biphasic stimulus, the centre electrode 304 in the hexagon 302 is connected to the power sources associated with its respective hexagon. Return path electrodes are connected to either a supply voltage or to a current or voltage sink. During the second charge recovery phase of biphasic stimulation, the electrical connections of the centre electrode and the return path are reversed.

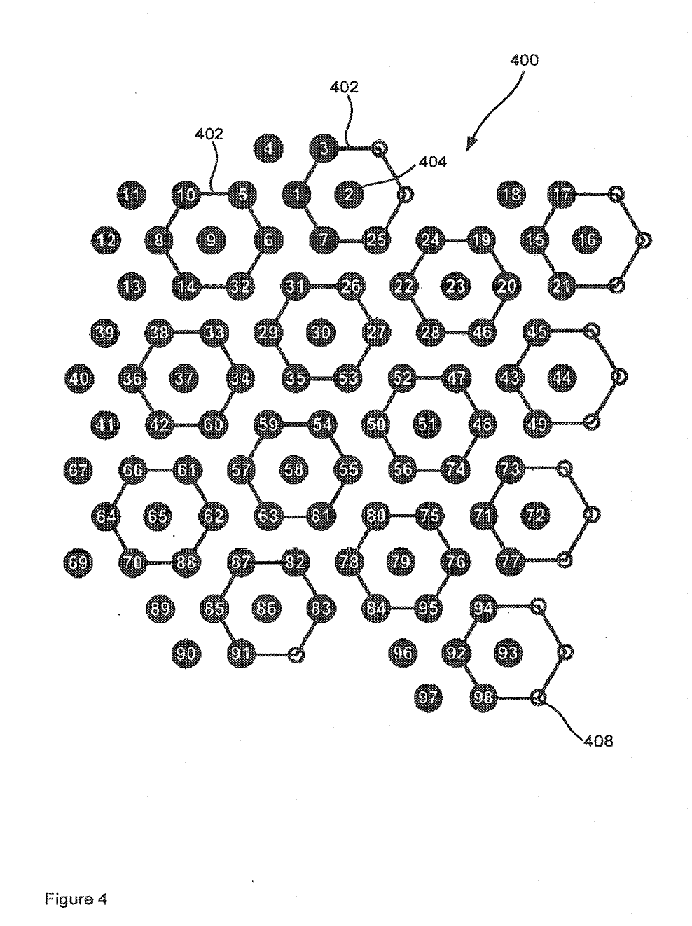

For different stimulating paradigms, different electrodes in the array 200 are selected to be the stimulating electrodes. This is done by superimposing different logical arrays on the electrode array 200. For example, repositioning the logical array to obtain hexagon array 400 shown in FIG. 4 ensures that different electrodes are placed at the centre of each hexagon 402, such as electrode 404. In logical array 400 the hexagons at the edge of the physical array 200 are incomplete and include unpopulated positions 408. By repositioning the logical array, there exist seven different ways to orient a hexagonal logical array on the electrode array 200, of which two ways are shown in FIGS. 3 and 4.

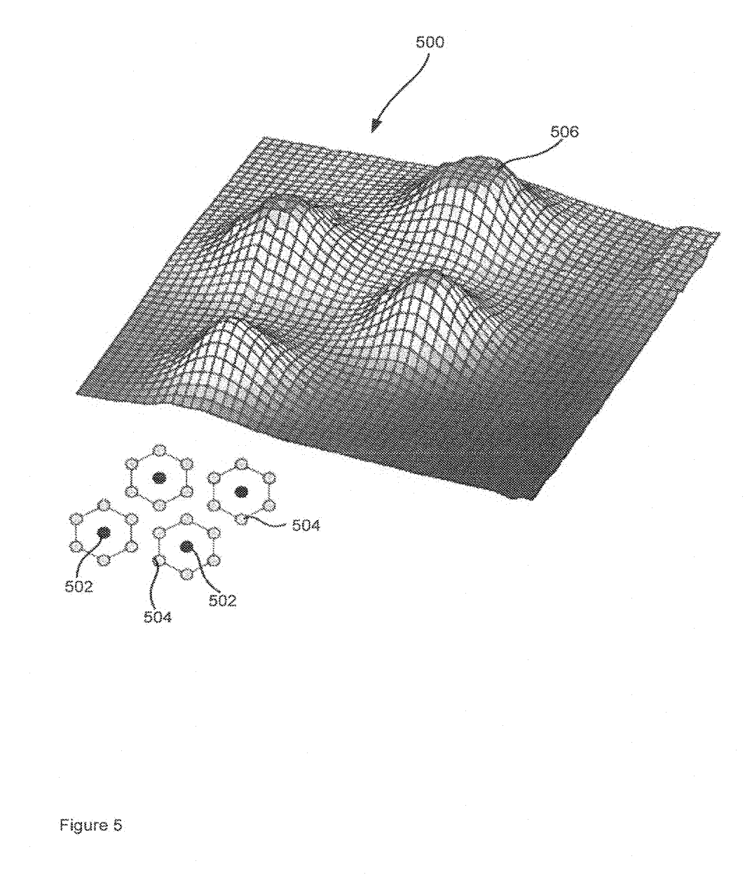

One consequence of arranging the electrodes in hexagonal groups is that each active electrode is surrounded by up to six electrodes that can function as return electrodes. When all or most of the six are used to collectively return the current delivered to the stimulating electrode then the electrodes surrounding the active electrode can be considered to be “guard electrodes”, or a “guard ring” because they limit the spatial distribution of the electrical field generated by the active electrode. FIG. 5 shows a measured voltage topography 500 resulting from the use of four stimulating electrodes 502 with guard electrodes 504. The discrete peaks 506 in the electrical field illustrate how the guard rings result in a limited area being stimulated by each electrode 502 so that little interference occurs between the stimulus from adjacent electrodes 502.

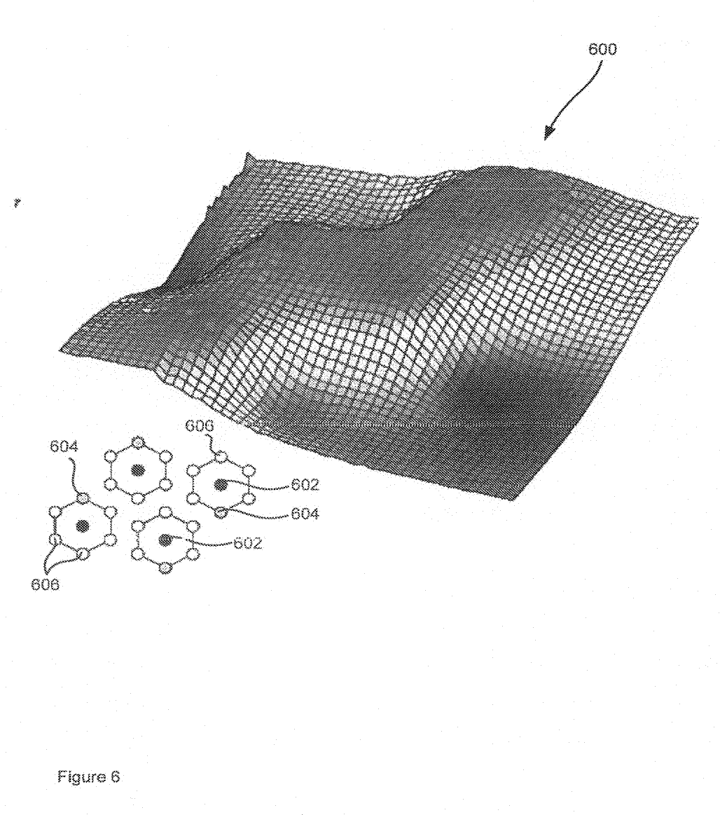

In contrast, FIG. 6 illustrates a measured voltage topography 600 of four stimulating electrodes 602 with a single hexagon electrode return electrode 604 in each of the four hexagons, while the remaining electrodes 606 are inactive. The interference between the electrical fields resulting from the four stimulating pulses can be seen in the topography 600, in which the peaks are not as distinct as the peaks 506 in FIG. 5.

In the arrangements illustrated in FIG. 5 and FIG. 6, the return paths are provided by electrodes that form part of the hexagons. These electrodes are called “bipolar electrodes” and they can be stimulating electrodes, or form part of the return path. They form “two poles” as opposed to the monopolar situation where there is a single pole involved in the electrical stimulation. The electrodes in the hexagonal patterns can also remain inactive if they are not used in the return path.

In a further arrangement, the central electrodes of the hexagons are used as stimulating electrodes, and a separate monopolar electrode that does not form part of the electrode array 200 provides the return path. This is illustrated as monopolar electrode 116 in FIG. 1. Other locations of monopolar electrode 116 may be contemplated and the system may have more than one monopolar electrode. FIG. 1 is merely an illustrative example of a location within the recipient's orbit.

In this arrangement, because all stimulating electrodes share the same return path there will generally be some interference between the electrical fields resulting from the stimulus of each stimulating electrode. Although this interference is not desirable, monopolar electrical stimulation does typically yield lower stimulation thresholds than other return path configurations. The stimulation threshold is the level of stimulation required in order to elicit action potentials from the neurons 118.

A monopolar return path is considered to be a return path provided by a monopolar electrode that is spaced at least multiple electrode diameters away from the stimulating electrode/s. In contrast, a bipolar return path is considered to be a return path provided by one or more electrodes that lie within the area of activation of the stimulating electrode array.

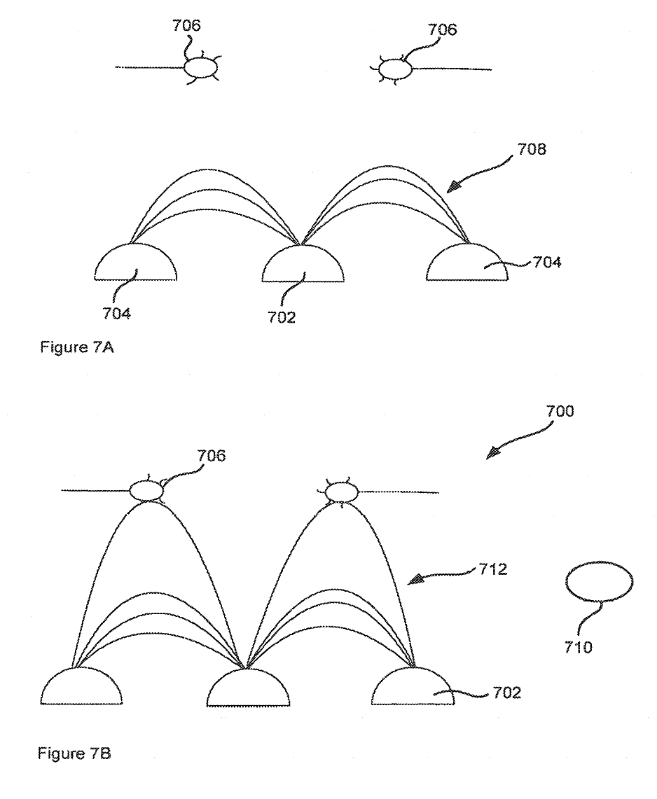

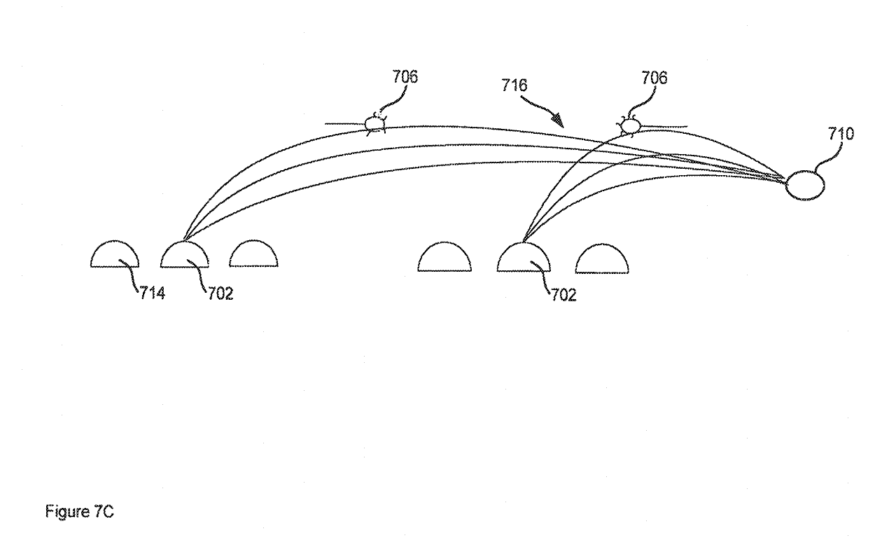

Referring to FIG. 7A, stimulating electrode 702 and return path electrodes 704 are positioned to lie along the interface between the choroid and the sclera, as described above with reference to FIG. 1. The neurons 706 that need to be stimulated lie in the neural retina of the eye. The hexagonal configuration using the guard ring return path as described above with reference to FIG. 5 (termed hexapolar stimulus) results in an increased concentration of electrical field for a given stimulation strength. As illustrated at 708, for an increasing distance from stimulating electrode 702, the density of the electrical field reduces to a greater extent than is needed to activate neurons 706. The stimulation threshold that will result in the electrical field 708 being strong enough to activate the neurons 706 is typically higher than for a configuration where the return path is provided through a monopolar electrode.

However, if a monopolar electrode 710 is added to the hexagonal configuration of FIG. 7A to form a hybrid configuration 700 as shown in FIG. 7B, then the addition of monopolar electrode 710 is thought to result in a local reshaping of the electrical field to provide a reshaped field 712 that is strong enough activate the neurons 706 even though a similar stimulation strength is being used. In other words, the stimulation threshold of the hybrid configuration is less than the stimulation threshold of the hexagonal guard ring configuration.

In FIG. 7C the monopolar electrode 710 provides the only return path when stimulation is applied via electrodes 702 and the “guard” electrodes 714 in the hexagons are inactive. This configuration results in an electrical field 716 with a low stimulation threshold but which suffers from crosstalk. For example, the right-hand neuron 706 may be affected by electrical fields associated with both of the stimulating electrodes 702.

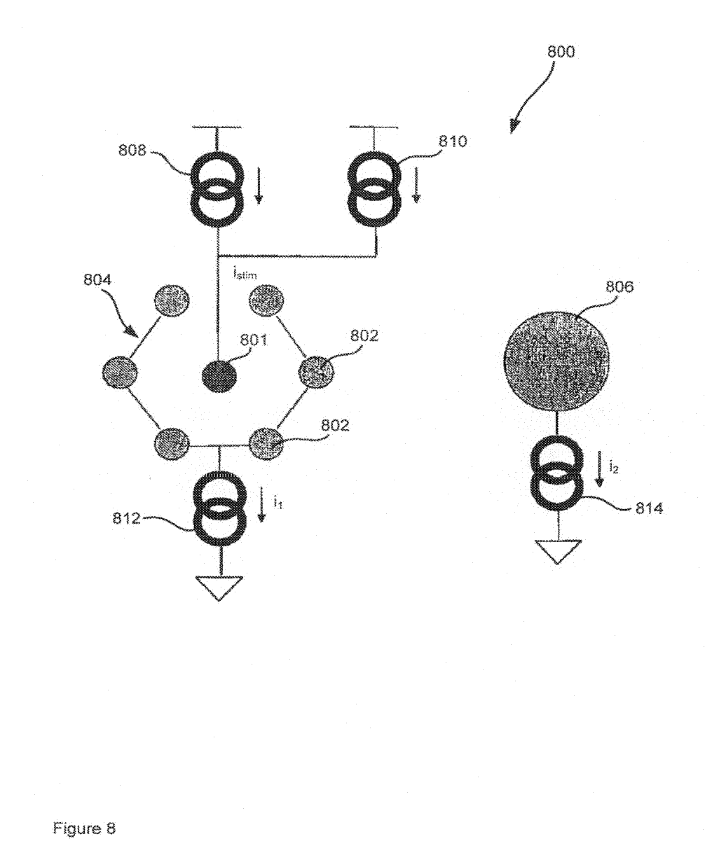

In the embodiment of a stimulation circuit 800 shown in FIG. 8, the stimulating current provided by stimulating electrode 801 is provided by current sources 808 and 810. In configuration 800 the return path of the stimulating current provided by stimulating electrode 801 is divided. One or more of the guard electrodes 802 provide part of the return path through current sink 812, and the remainder of the current returns through monopolar electrode 806 and current sink 814. This reduces the required stimulation threshold that is needed to stimulate the neurons because of the use of the monopolar electrode 806. However, the configuration 800 also provides the benefits of charge concentration from the guard electrodes 802 in the hexagon 804.

In this embodiment, the return current through the guard electrodes 802 is i₁ and is divided approximately equally through each of these electrodes. The return current through current sink 814 is i₂. When i₁=0 and i₂>0, all current that is injected from the stimulating electrode 801 returns via the monopolar electrode 806. In this situation one would anticipate the lowest stimulation threshold to be observed. When i₂=0 and i₁>0, all current returns via one or more of the hexagon's bipolar electrodes 802. When all six of these electrodes 802 act as return electrodes, as discussed with reference to FIG. 5 for example, then stimulation can occur from multiple sites having respective guard rings simultaneously without significant cross-talk between these sites.

In this embodiment, the stimulating current is divided such that the benefits of threshold reduction are realised by way of the monopolar return path, and the benefits of charge containment through the use of the guard ring electrodes 802 are realised at the same time. The stimulation current is therefore given by iₛₜᵢₘ=i₁+i₂.

Different ratios of i₁:i₂ will result in different trade-offs between low stimulation threshold and charge containment, and this depends (amongst other factors) on the diameter of the electrodes that are used. Other factors that influence the ratio used include how far apart the electrodes are from one another because the further apart they are, the less the benefit that may be obtained by the use of the guard ring. Another factor is the thickness of the choroid, which influences the field required.

For example, i₁ may be between 10 and 50% of the total return current while i₂ is between 90 and 50%. In one embodiment, the return current through the monopolar electrode 806 i₂ is approximately 75% of the total return current while the return current through the guard electrodes 802 i₁ is approximately 25% of the return current.

In another embodiment there may be additional return paths, for example provided by an additional monopolar electrode. FIG. 8 shows a single hexagon 804. It will be appreciated that the electrode array may include multiple hexagons.

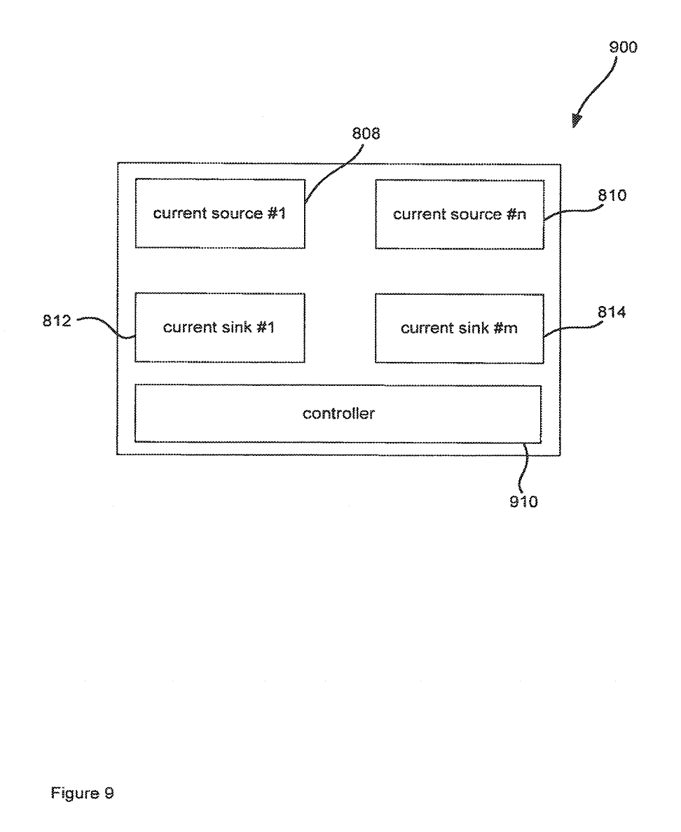

FIG. 9 shows a schematic diagram of the circuitry 900 used to implement the hybrid return path. This circuitry would typically be implemented in the electronics capsule 112 shown in FIG. 1. The circuitry 900 includes at least one current source 808, 810 for association with the stimulating electrodes of the electrode array. The circuitry 900 further includes at least one current sink 812, 814 for association with the return electrode or return path. For example current sink number 1 may be associated with the guard electrons of a first hexagon while current sink number 2 may be associated with a monopolar return electrode. The circuitry further includes a controller 910 that controls the ratio of the current returned via the respective current return paths used in a hybrid configuration. The controller may be adjustable so as to vary the ratio of the return currents.

The current sources and current sinks may be provided in a push-pull configuration. For example current source 808 and current sink 812 may be associated with one another, and similarly current source 810 may be associated with current sink 814. The paired sources and sinks may be associated with respective constant-current digital to analogue converters (DACs). If a matched push-pull configuration is used for the current sources and sinks, then an equal amount of current injected by the current source of any one DAC is drawn by the matching sink for that DAC (for example source 808 and sink 812). During concurrent stimulation in which multiple DACs are active, this ensures that during the anodic phase, although multiple DACs are stimulating through the monopolar return 806, only the previously sourced amount of current is returned to the retinal electrodes.

In FIG. 8 there are two independent current sources connected to stimulating electrode 801, permitting a quasi-monopolar stimulation (i.e. combining monopolar and hexapolar stimulation). Alternatively, by injecting current using just one of the two DACs, pure monopolar stimulation or pure hexapolar stimulation may be used. For example, pure hexapolar stimulation may be obtained by using the DAC for current source 808 and current sink 812. Similarly, pure monopolar stimulation may be achieved by using the DAC for current source 810 and current sink 814. Using both DACs simultaneously increases the total current through the stimulating electrode 801.

Experiments were conducted to study the effects of different ratios of on the stimulation threshold. In these experiments, a 24-electrode array comprising stimulating platinum electrodes, each of 380 μm in diameter, was used. Of the 24 electrodes, 10 electrodes formed complete hexagons, such as hexagons 302 as illustrated in FIG. 3, whereas the rest of the electrodes were at edges of the array, such as those occupying unpopulated positions 408 as illustrated in FIG. 4. The array was implanted into the suprachoroidal space of the feline eye (with the experiments conducted with n=6 eyes from a total of 5 animals). Following a craniotomy and durotomy, a 10*10 penetrating array (Utah Array, Blackrock Microsystems, Utah, USA) was inserted and connected to a RZ2 multi-channel data acquisition system (Tucker-Davis Technologies, Florida, USA). The retina was stimulated using charge-balanced, constant current, biphasic stimuli with a constant phase time of 500 μs and the resulting cortical activity was recorded. A return current i₁ of 700 μA through the guard electrodes 802 (termed hexapolar stimulus) was superimposed with a return current i₂ of 0 μA, 37 μA, 72 μA and 108 μA through the monopolar electrode 806 (termed monopolar stimulus). The recordings were filtered and spike counting was performed offline using Matlab (The Mathworks, Inc., USA), and sigmoid curves were fitted to model the effect of increasing stimulation current on the cortical activity. The midpoint on the slope (P50) of the sigmoid was chosen as an arbitrary indication of threshold and the results compared.

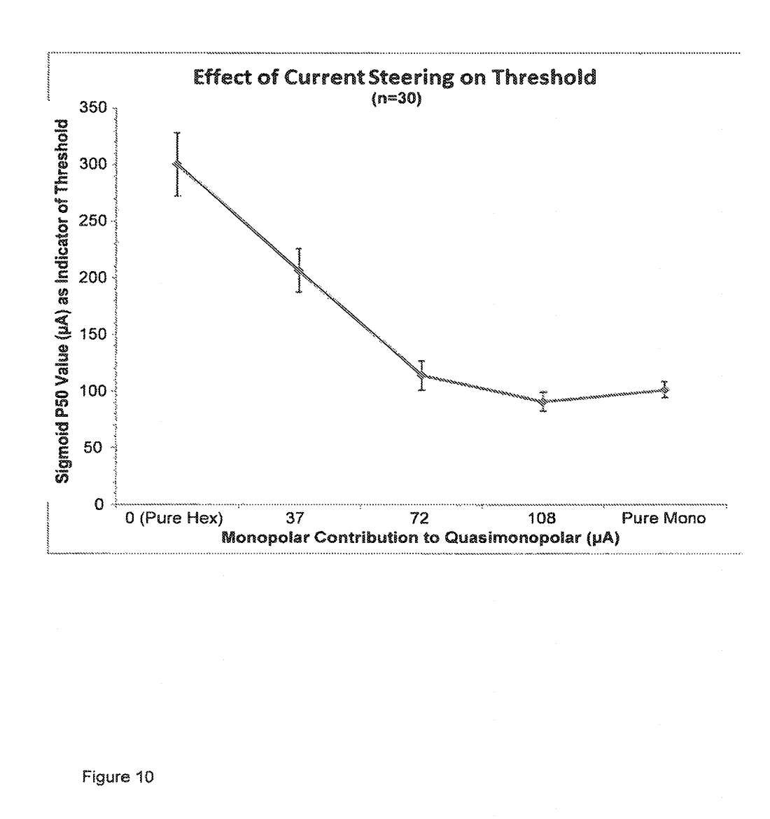

Referring to the experimental results illustrated in FIG. 10, the first data point where the monopolar current i₂ is 0 μA represents a pure hexapolar stimulus, where all stimulation current returns through the guard electrodes 802 and none returns through the monopolar electrode 806. The stimulation threshold for a pure hexapolar stimulus was determined to be 300 μA±28 μA (standard errors are indicated by the bars in FIG. 10). With the addition of 37 μA of monopolar stimulus represented by the second data point, the stimulation threshold was found to drop by almost a third, to 206 μA±19 μA. At the third data point, 72 μA of monopolar stimulus resulted in a further drop to 113 μA±13 μA. At the fourth data point, 108 μA of monopolar stimulus resulted in a threshold of 90 μA±8 μA. The fifth data point represents a pure monopolar stimulus (i.e. i₁ is 0), which resulted in a stimulation threshold of 101 μA±7 μA. In these results the mean stimulation threshold of the fifth data point (that is, for a pure monopolar stimulus) is slightly higher than that of the fourth data point. This is thought to be a data processing artefact and in general it is anticipated that the threshold will be lowest for pure monopolar stimulation. These results indicate that combining monopolar and hexapolar stimuli yields lower stimulation thresholds than using a hexapolar stimulus alone. This is consistent with the presence of monopolar and hexapolar fields around the electrodes, and confirms a superposition effect wherein higher charge density elicits action potentials for a significantly lower overall charge.

Experiments were also conducted to study the effects of different ratios of i₂ on charge containment. A best cortical electrode (BCE) was chosen as the electrode with the highest maximum spike rate and the lowest P50 value. Using the spike counting data collected above, the probability of a spike occurring was calculated on the best cortical electrode (BCE), and then the probability of a spike occurring simultaneously in every other site was calculated using:

$$P ( {El}_{y} \vert BCE ) = \frac{P ( {El}_{x} ⋂ BCE )}{P ( BCE )}$$

where P(Elₓ|BCE) is the probability of a spike occurring at a given site Elₓ given that it also occurred at the BCE, P(Elₓ∩BCE) is the probability of a spike occurring at a site Elₓ and BCE simultaneously, and P(BCE) is the probability of a spike occurring on the BCE.

In these experiments, using these values, the specific case where P(BCE) attains a maximum value was observed to maximise the spread of the electrical field, and the probability of spikes occurring across all electrodes was observed. If P(Elₓ|BCE) was greater than 0.5, then the site was considered “active” and that site was counted, otherwise it was ignored. The channels of all stimulation strategies were then normalised with respect to the channel count of a pure monopolar stimulus to eliminate bias introduced by the placement of the stimulating electrode.

Experimental results are illustrated in FIG. 11, which are normalised to the case of a pure monopolar stimulus. The guard electrodes in the pure hexapolar arrangement (i₂=0 μA) recruited (54±13) % of the number of sites. With the addition of i₂=37 μA of monopolar stimulus, the recruitment was (42±7) % of the number of sites. With i₂=72 μA and 108 μA of monopolar stimulus, the recruitment was (44±4)% and (55±6)% respectively. FIG. 11 shows that quasi-monopolar stimulus offers significant activation containment with respect to pure monopolar stimulation, and approximates that of hexapolar stimulation.

Multi-Source Stimulation

In a further arrangement, a stimulation system uses monopolar and hexapolar fields generated by different sources. In this system one or more electrodes are used in a pure hexapolar configuration to provide local stimulation, and at least one electrode is used in a monopolar or a quasi-monopolar configuration that superimposes a hexapolar stimulation and a monopolar stimulation. The monopolar or quasi-monopolar source provides a sub-threshold charge. The advantages of sub-threshold monopolar stimulation are found to benefit nearby, purely hexapolar electrodes. For example, the benefits of sub-threshold monopolar stimulation may be detected with a hexapolar field up to three electrodes away from the monopolar stimulation source.

An example is shown in FIG. 12A, in which there are three hexagons of electrodes 804, 820 and 830. Hexagon 804 consists of stimulating electrode 801 surrounded by six guard electrodes 802. Hexagon 820 consists of stimulating electrode 821 surrounded by six guard electrodes 822. Hexagon 830 consists of stimulating electrode 831 surrounded by six guard electrodes 832. In FIG. 12 the hexagons are depicted separately to illustrate their functioning, rather than their physical configuration relative to one another. In practice the three hexagons may be part of an array like that shown in FIG. 2.

Electrode 801 operates in a quasi-monopolar mode. Two independent constant current sources 808, 810 are connected to electrode 801. The current sink 812, associated with current source 808, is connected to the six guard electrodes 802 that surround stimulating electrode 801. The current sink 814, which is associated with current source 810 in a push-pull configuration, is connected to the distant monopolar electrode 806.

Electrode 821 operates in a hexapolar mode. Current source 818 is connected to the stimulating electrode 821. The current sink 824 associated with current source 818 is connected to the six guard electrodes 822 that surround stimulating electrode 821.

Likewise, electrode 831 operates in a hexapolar mode. Current source 828 is connected to the stimulating electrode 831. The current sink 834 associated with current source 828 is connected to the six guard electrodes 832 that surround stimulating electrode 831.

In this arrangement, only electrode 801 carries a combined current from two current sources. Electrodes 821, 831 are each connected to one current source.

In a further arrangement, shown in FIG. 13A, the electrodes are used in either a hexapolar mode or a monopolar mode, but not both.

Electrodes 821 and 831 are used in a hexapolar configuration, as in the arrangement of FIG. 12. Electrode 851 is used in a pure monopolar configuration. The DAC for current source 850 and current sink 852 is used. Current source 850 is connected to electrode 851. Although electrode 851 is surrounded by six electrodes 854, this hexagon of potential guard electrodes is not connected to a return path. Instead, the current sink 852 is connected to the monopolar electrode 806.

FIG. 13A shows an example in which a monopolar stimulation source 860 is used to provide a sub-threshold monopolar field and two other local sources 820, 830 are used in a hexapolar configuration to provide local neural stimulation. Different numbers of electrodes may be used, such that a plurality of hexapolar stimulation electrodes are interspersed with monopolar “field generators” that provide stimulation at a current level which is sub-threshold for their location. The sub-threshold monopolar field causes no retinal activation and therefore no loss of activation focus is expected.

The arrangements of FIGS. 12A and 13A provide a sub-threshold charge from one or more electrodes in the vicinity of excitable neural tissue. Local electrodes provide additional charge to reach the local threshold for stimulation. The arrangements reduce the burden of charge-carrying capacity on the local electrodes. This configuration is thought to facilitate the use of smaller electrodes. Consequently, electrode arrays may be more densely packed. In the arrangement of FIG. 12A the stimulating electrode 801 is capable of carrying a larger charge and is hence physically larger than electrodes that carry only a monopolar or hexapolar current. The larger size of electrodes such as electrode 801 implies a lower electrode density. In contrast, in the arrangement of FIG. 13A the electrodes 851, 821 and 831 need a relatively lower charge-carrying capacity than electrode 801. This enables a denser packing of the electrode array.

FIGS. 12B and 13B are schematic diagrams that illustrate the hexapolar and monopolar fields generated in the arrangements of FIGS. 12A and 13A respectively. There are three electrodes 821, 801 and 831 located near neurons 921, 923 and 925 respectively. Electrode 801, which has two current sources 808, 810 connected to it, generates a monopolar field 960 and a hexapolar field 953. The monopolar contribution 960 provides a sub-threshold level that does not stimulate any of the three neurons 921, 923, 925. However, all three neuron sites benefit from the monopolar field. The hexapolar fields 951, 953 and 955 generated by the electrodes 821, 801, 831 stimulate the respective neurons 921, 923, 925.

FIG. 13B is similar, except that the central electrode 851 operates in a pure monopolar mode to provide monopolar field 960. Thus, electrode 851 does not elicit a response from the middle neuron 923 and accordingly carries less charge or current. The other sites involved in the stimulation (which could be more than the small number illustrated here) may elicit responses from their associated neurons 921, 925 at a lower threshold because of the monopolar field 960.

The foregoing arrangements described with reference to FIGS. 1 to 13B relate to a planar array of electrodes used in a visual prosthesis for the treatment of blindness. Other applications may also benefit from a combination of a sub-threshold charge supplemented by one or more local electrodes to stimulate excitable tissue. For example, in deep brain stimulation of the sub-thalamic nucleus, or stimulation of the auditory system via the cochlea, an array of electrodes assembled in a longitudinal fashion is implanted. In such cases, benefits of a similar nature to those described above may be achieved. This includes the capacity to reduce the perceptual or physiological threshold of a given electrode by sharing the total electrical charge required in order to elicit a response from a single electrode pair or multiple sets of electrodes simultaneously.

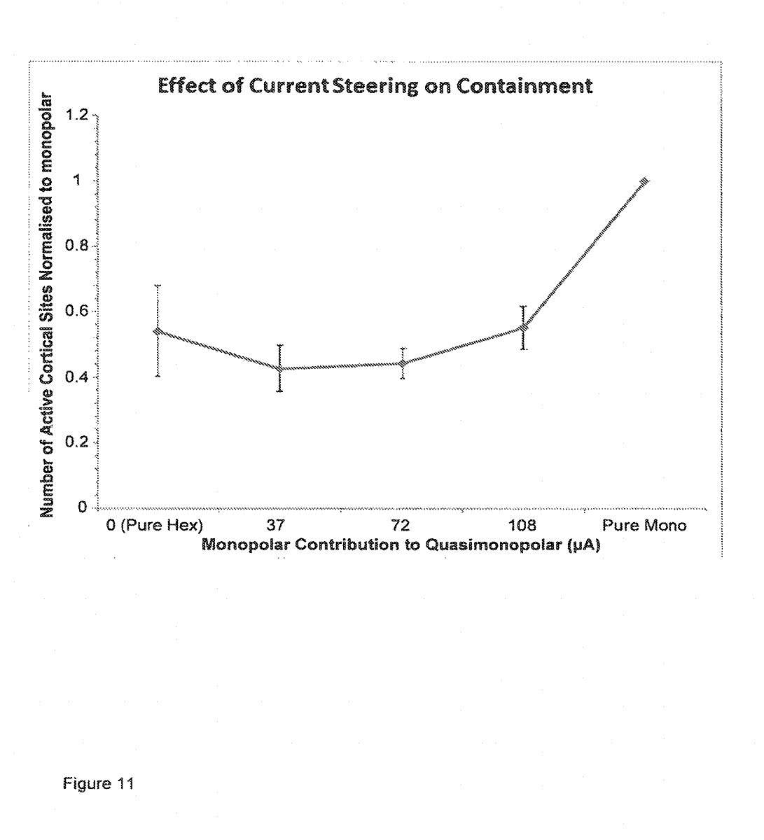

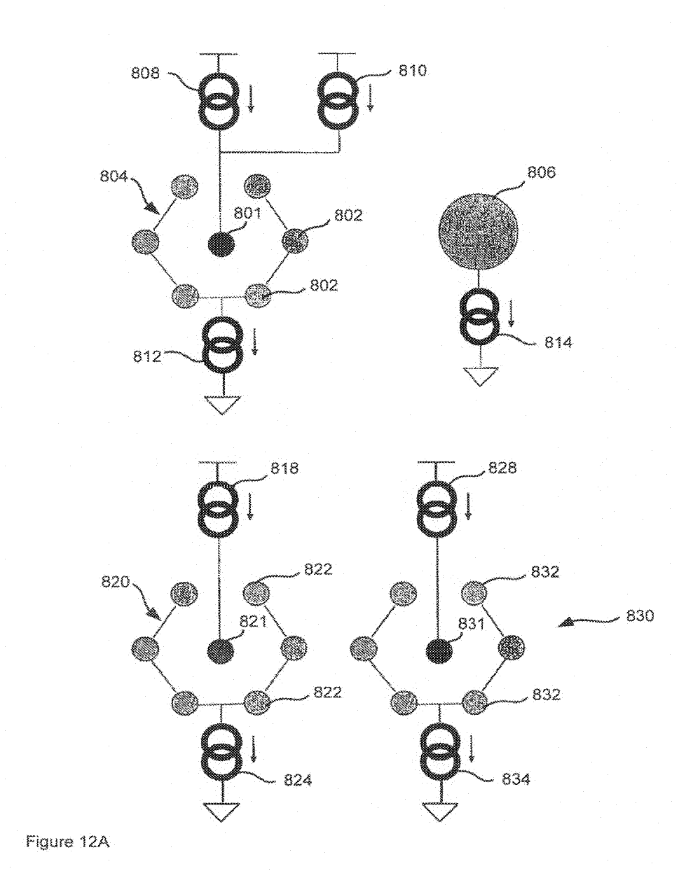

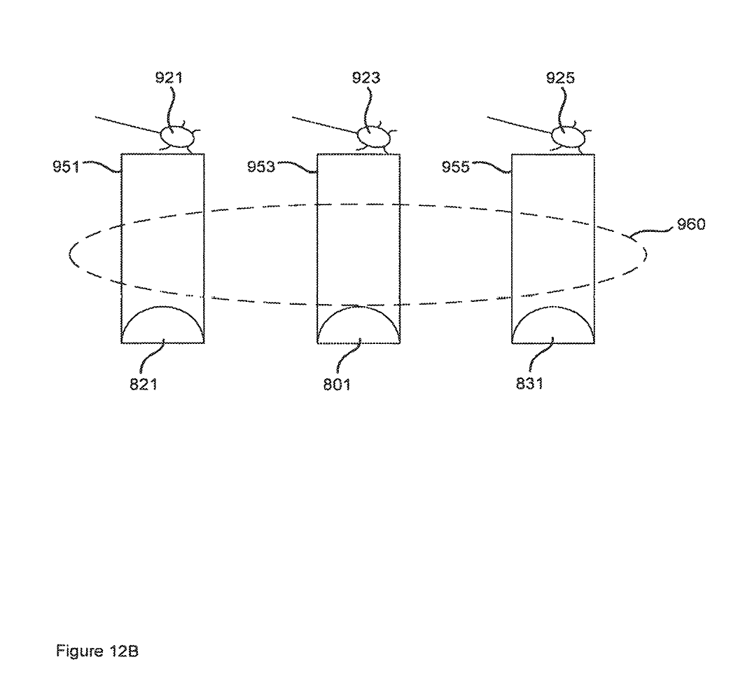

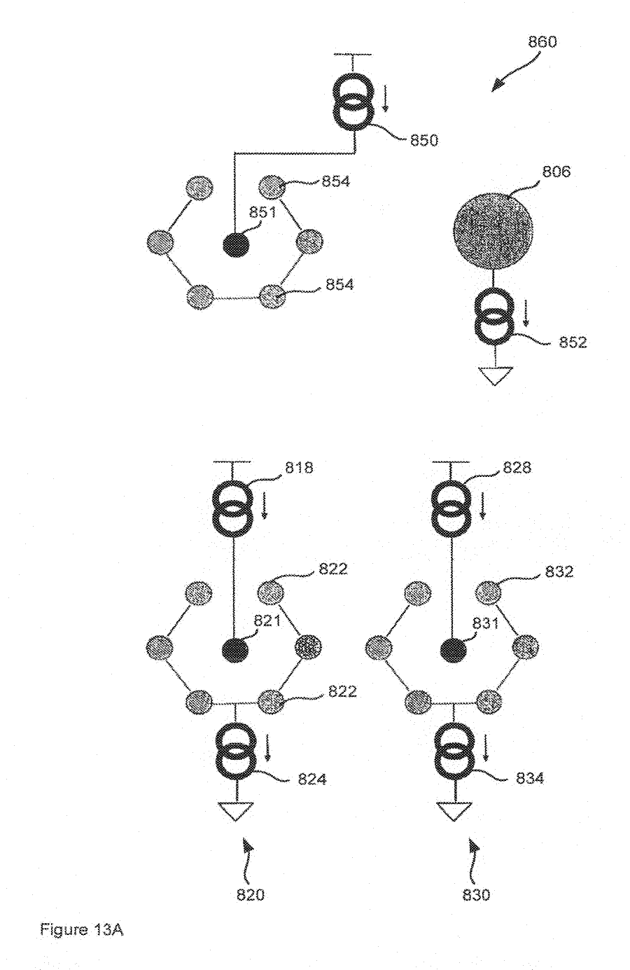

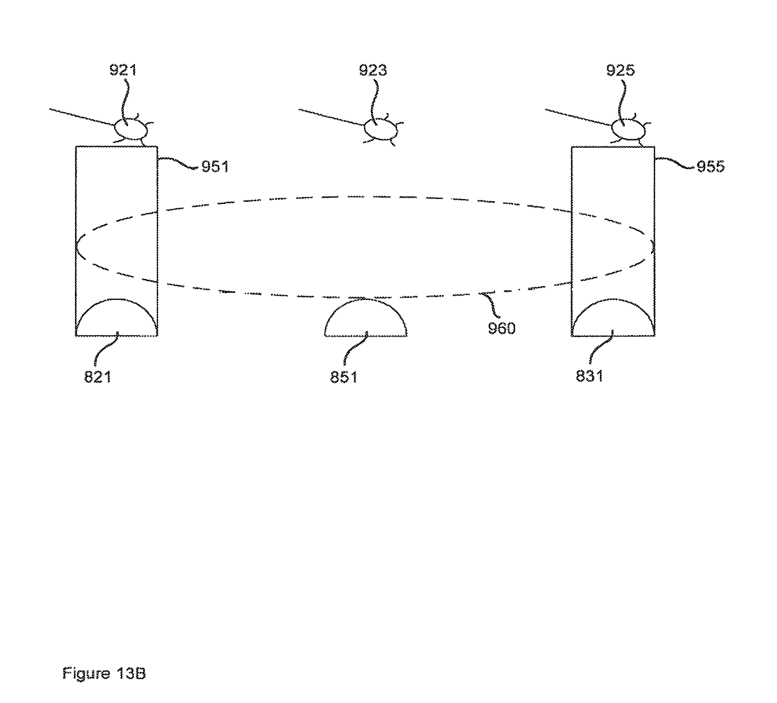

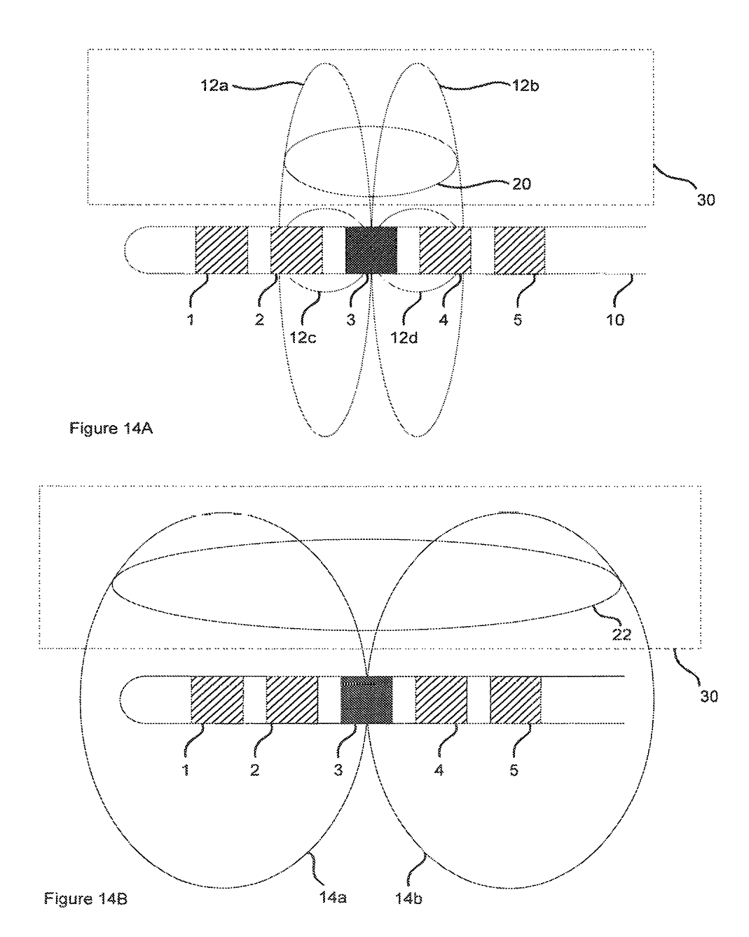

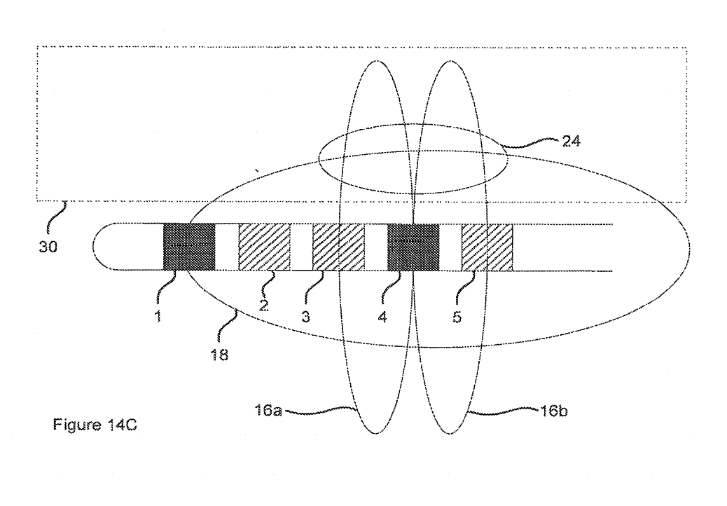

FIGS. 14A, 14B and 14C show an illustrative example of a longitudinal electrode array 10 deployed in the vicinity of target tissue 30. The illustrated array has five electrodes 1, 2, 3, 4, 5 although in practise the array 10 may have a larger number of electrodes. FIG. 14A illustrates a “tri-polar” application of the longitudinal electrode array 10 using three electrodes 2, 3, 4. In this mode the return path of stimulation is via a single or a plurality of electrodes within the array 10 or nearby the array 10. “Nearby” is used in contrast to a monopolar electrode that is “far” away from the stimulating electrode, for instance spaced at least multiple electrode diameters away from the stimulating electrode/s. Stimuli 12a-d are being delivered from electrode 3, thereby activating region 20 of the target tissue 30. The schematic diagram shows multiple stimuli (e.g. circle 12c and ellipse 12a) to indicate the required strength of the stimulus. The fact that both the circle and ellipse are shown (as opposed to only one in FIGS. 14B and 14C) indicates that a greater amount of charge is required to penetrate into and activate the tissue 20, compared with the arrangement described below with reference to FIGS. 14B and 14C.

The tri-polar arrangement of FIG. 14A has a relatively high charge requirement, low penetration and high shunting, compared with the monopolar arrangement described below with reference to FIG. 14B.

FIG. 14B illustrates the use of a monopolar field. In addition to the electrode array 10, a monopolar electrode (not shown) is implanted in the recipient's tissue. A broad monopolar field is generated, represented by the ellipses 14a and 14b representing monopolar stimulation via electrode 3 with a return path through the monopolar electrode. In this monopolar arrangement tissue 22 is recruited, i.e. affected by but not necessarily activated by the broad monopolar field. Activation means sufficient depolarisation to elicit a response upon reaching a threshold, and recruitment indicates depolarised tissue that may or may not have reached a threshold of activation.

In comparison with the tri-polar arrangement of FIG. 14A, the monopolar field has relatively high penetration, high spread and requires relatively low charge.

FIG. 14C shows the use of monopolar stimulation in conjunction with the localised stimulation provided by the electrodes of the longitudinal electrode array. Electrode 1 and the monopolar electrode provide a monopolar stimulation 18 that in general use is a sub-threshold field although it is possible that a threshold of activation may be reached.

Concurrently, electrodes 3,4,5 are used in a tri-polar stimulation with local return paths, generating local stimulus 16a, 16b. Tissue 24 is activated where the local stimulus 16a, 16b and the monopolar stimulus 18 overlap. The presence of the monopolar field 18 reduces the amount of current required to be delivered from the local stimulus. Consequently, the total current delivered from (or to) any single electrode is reduced, thereby allowing the electrode's geometric size to be reduced, or the addition of a greater level of safety to existing electrode geometries.

Compared with the arrangements of FIGS. 14A and B, the concurrent arrangement of FIG. 14C has low to medium charge requirements, a medium to high penetration and a high phosphine focus.

It will be understood that the invention disclosed and defined in this specification extends to all alternative combinations of two or more of the individual features mentioned or evident from the text or drawings. All of these different combinations constitute various alternative aspects of the invention.

1-11. (canceled)

12. A system for stimulating excitable tissue, comprising:
a monopolar stimulation source comprising:
a first monopolar electrode connected to at least a first current source, and
a second monopolar electrode distant from the first monopolar electrode, the second monopolar electrode connected to a first current sink, the first current sink associated in a push-pull configuration with the first current source for providing a monopolar electrical return path, the monopolar stimulation source configured to generate a first sub-threshold field in the vicinity of the excitable tissue; and
a bipolar stimulation source comprising:
a first bipolar electrode connected to at least a second current source different to the first current source, and
a second bipolar electrode proximate to the first bipolar electrode and distant from the second monopolar electrode, the second bipolar electrode connected to a second current sink different to the first current sink, the second current sink associated in a push-pull configuration with the second current source for providing a bipolar electrical return path, the bipolar stimulation source configured to generate a second sub-threshold field in the vicinity of the excitable tissue, the bipolar stimulation source configured to generate the second sub-threshold field as a local field simultaneously with the first sub-threshold field,
wherein, in combination the first sub-threshold field and the second sub-threshold field exceeds a threshold at which activation of the excitable tissue occurs.

13. The system of claim 12, further comprising a plurality of bipolar stimulation sources that each generate a respective second sub-threshold field, wherein each second sub-threshold field in combination with the first sub-threshold field exceeds the threshold of the excitable tissue at a respective stimulation site.

14. The system of claim 13, wherein the plurality of bipolar stimulation sources comprises an electrode array with a plurality of first bipolar electrodes each having at least one associated bipolar return path.

15. The system of claim 14, wherein the electrode array is planar.

16. The system of claim 15, wherein the planar electrode array comprises a plurality of second bipolar electrodes spatially arranged around respective first bipolar electrodes.

17. The system of claim 14, wherein the electrode array is longitudinal.

18. The system of claim 12, wherein the first monopolar electrode and the first bipolar electrode are the same electrode.

19. The system of claim 12, wherein the first monopolar electrode and the first bipolar electrode are different electrodes, spaced apart from each other.

20. The system of claim 12, wherein the second monopolar electrode is spaced at least multiple electrode diameters away from the first monopolar electrode and wherein the second bipolar electrode lies within the area of activation of the first bipolar electrode.

21. A neural prosthesis comprising:
an electrode array comprising a plurality of stimulating electrodes each having at least one associated bipolar return electrode; and
a monopolar return electrode;
a plurality of bipolar electrical return paths associated with the respective bipolar return electrodes; and
a monopolar electrical return path associated with the monopolar return electrode;
wherein, in use, the plurality of stimulating electrodes provide stimulating currents to the tissue of a recipient; and for at least one stimulating electrode a total return current is divided between a first current in the associated bipolar electrical return path and a monopolar current in the monopolar electrical return path.

22. The neural prosthesis of claim 21 wherein the stimulating electrodes each have a plurality of bipolar return electrodes spatially arranged around the associated stimulating electrode and wherein the bipolar electrical return path for the associated stimulating electrode is associated with the plurality of bipolar return electrodes.

23. The neural prosthesis of claim 21 further comprising a controller to set relative magnitudes of the bipolar return currents and the monopolar return current.

24. A method for stimulating excitable tissue, comprising:
generating, with a monopolar stimulation source, a first sub-threshold field in the vicinity of the excitable tissue, the first sub-threshold field being below a threshold at which activation of the excitable tissue occurs, the monopolar stimulation source comprising:
a first monopolar electrode connected to at least a first current source, and
a second monopolar electrode distant from the first monopolar electrode, the second monopolar electrode connected to a first current sink,
wherein generating the first sub-threshold field comprises operating the first current sink in a push-pull configuration with the first current source to provide a monopolar electrical return path; and
generating, with a bipolar stimulation source, a second sub-threshold field in the vicinity of the excitable tissue, the second sub-threshold field generated as a local field simultaneously with the first sub-threshold field, the bipolar stimulation source comprising:
a first bipolar electrode connected to at least a second current source different to the first current source, and
a second bipolar electrode proximate to the first bipolar electrode and distant from the second monopolar electrode, the second bipolar electrode connected to a second current sink different to the first current sink,
wherein generating the second sub-threshold field comprises operating the second current sink in a push-pull configuration with the second current source to provide a bipolar electrical return path;
wherein in combination the first sub-threshold field and the second sub-threshold field exceeds the threshold at which activation of the excitable tissue occurs.

25. The method of claim 24, wherein generating the second sub-threshold field comprises:
generating a plurality of sub-threshold local fields with a plurality of respective local stimulation sources, wherein each sub-threshold local field in combination with the first sub-threshold field exceeds the threshold of the excitable tissue at a respective stimulation site.

26. The method of claim 24, wherein the first monopolar electrode and the first bipolar electrode are the same electrode.

27. The method of claim 24, wherein the first monopolar electrode and the first bipolar electrode are different electrodes, spaced apart from each other.

28. The method of claim 24, wherein the second monopolar electrode is spaced at least multiple electrode diameters away from the first monopolar electrode and wherein the second bipolar electrode lies within the area of activation of the first bipolar electrode.
